(19) United States
(12) Patent Application Publication
(10) Pub. No.: US 2010/0061947 A1
(43) Pub. Date: Mar. 11, 2010

(54) UV PROTECTIVE COSMETIC PRODUCT INCORPORATING TITANIUM DIOXIDE AND TRANSPARENT IRON OXIDE

(76) Inventors: David Schlossman, Short Hills, NJ (US); Edward Bartholomey, Rosedale, MD (US); Carl Orr, Scotch Plains, NJ (US)

Correspondence Address: THOMPSON HINE L.L.P.;Intellectual Property Group, P.O. BOX 8801, DAYTON, OH, 45401-8801, US

(21) Appl. No.: 12/515,395

(22) PCT Filed: Nov. 15, 2007
(86) PCT No.: PCT/US07/84883

§ 371 Date: Jun. 26, 2009

Related U.S. Application Data
(60) Provisional application No. 60/859,591, filed on Nov. 17, 2006.

(51) Int. Cl. A61K 8/29; A61K 8/18; A61K 8/27; A61Q 17/04

(52) U.S. Cl. 424/59

(57) ABSTRACT

A cosmetic composition comprises a transparent substantially colorless pigment. The substantially colorless pigment comprises a first group of particles sufficiently small to be substantially transparent to visible light and substantially opaque to ultraviolet B light. The substantially colorless pigment further comprises a second group of particles sufficiently small to be minimally reflective to visible light and substantially opaque to ultraviolet A light. A second group of particles results in minimal visible reflection by the cosmetic composition. A substantially transparent coloring pigment substantially transmits visible light and reflects a substantial portion of incident ultraviolet A light to impart color to tint visible light reflected by the cosmetic composition when they cosmetic composition is disposed over the skin. These materials are maintained in a carrier vehicle.

### CROSS REFERENCE TO RELATED APPLICATION

This application claims the priority of Provisional Application Ser. No. 60/859,591 filed Nov. 17, 2006.

### TECHNICAL FIELD

The present invention relates to compositions having balanced UVA and UVB protection properties and more particularly to UV protective cosmetic products incorporating titanium dioxide and transparent iron oxide.

### BACKGROUND OF THE INVENTION

In 2007, there will be more than one million new cases of skin cancer reported of which it is estimated that 90 percent could have been prevented by better protection from the sun. During 2002, 44,582 cases of malignant melanoma were diagnosed in the United States alone. Most Americans do not adequately protect themselves from ultraviolet exposure.

Moreover, individuals receive 50 to 80 percent of their lifetime ultraviolet exposure by the age of eighteen. Thus, children and young people need to be better educated about sun damage. New and cosmetically attractive products would be very helpful in order to realize badly needed changes in behavior.

The situation is compounded by the fact that consumers cannot completely rely on sun protection factor (SPF) ratings, because the same are primarily tied to ultraviolet B light. At one time, it was believed that ultraviolet B light was the primary cause of skin damage. Certainly, reddening and sunburn are largely caused by ultraviolet B exposure. However, while it was not initially recognized, ultraviolet A exposure, in addition to tanning, likely causes long-term damage, including skin cancer and premature aging. Ultraviolet A light is in the range between 400 nm and going down to 320 nm. Ultraviolet B light begins about 320 nm and goes down to about 290 nm. The differences in wavelength are key to providing protection from the sun.

One effective way of reducing exposure to sunlight is the use of titanium dioxide and zinc oxide based sunscreens. The earliest sunscreens involved pigment grade materials and thus appeared like smears of chalk on the skin. Starting in the 1980s, particle manufacturers began to develop very fine particulate sunscreen materials. Typically, today, sun tanning lotions incorporating, for example, 10-150 nm titanium dioxide or zinc oxide are used to achieve a measure of protection from the sun.

References to particle size within this specification refer to the shortest dimension of a pigment particle. For example, if a pigment contains acicular particles which are 20 nm×100 nm, such particles are referred to as having a size of 20 nm. Moreover, references to particle size refer to the primary particle size of the powder ingredient. In various compositions, there may be some measure of conglomeration which would result in conglomeration sizes (i.e. secondary particle sizes) which are larger. Likewise, references to particle size are to the average size of the shortest dimension for an ingredient, as is the custom in the industry.

EP06164522 to Boots relates to 15 nm and 35 nm to about 50 nm titanium dioxide particles which can protect the skin against UVA and UVB light. The sunscreen products that are claimed therein are to some extent transparent to visible light. Similarly, other formulations providing broad spectrum protection are focused on transparency. For example, the prior art attempts to achieve broad spectrum protection by incorporating low refractive index pigment zinc oxide in combination with titanium dioxide.

Advantageously, as particle sizes become smaller, their chalky appearance becomes less and less noticeable, until they become substantially transparent, provided the particles are small enough and/or their concentration in the final product is not too high. Generally, as the particle size becomes much smaller than the wavelength of visible light, particles become invisible. Relatively high SPF products may be prepared using such particulate sunscreens. However, as the particles become substantially invisible, their ability to protect from ultraviolet A light is increasingly compromised.

### SUMMARY OF THE INVENTION

In accordance with the invention, novel cosmetic products incorporating titanium dioxide and transparent iron oxide are provided.

The invention contemplates sunscreen formulation involving the use of particles of different sizes. For example, particles in the 10 nm range may be very effective in reducing ultraviolet B light. Moreover, because they are very transparent to the eye, they may be incorporated in relatively large quantities into a sunscreen formulation. Thus, a high SPF sunscreen formulation may be achieved with minimal whitening of the skin. In addition, in accordance with the invention, larger particles, for example, 50 nm and 60 nm are also incorporated in the formulation with the object of longer wavelengths of light, for example, light in the ultraviolet A range. In accordance with the invention, it has been discovered that in such formulations, 60 nm particles are particularly effective in providing protection against ultraviolet A light.

However the aesthetics of colorless, for example zinc oxide or titanium dioxide, larger particles if used alone, particularly 60 nm particles, leave something to be desired. They tend to impart a relatively chalky appearance to the skin, and this degree of whitening may not be acceptable to many consumers. For this reason, many products which boast high sun protection factors and high transparency may provide relatively poor protection against ultraviolet A light.

Currently, protection against ultraviolet light is measured using the so-called “PA” (or “PFA”) rating, referring to protection against ultraviolet A light. Current thinking is that the PA rating be at least one third that of the SPF rating. However, in many higher SPF rated products, the PA rating may only be about three, as would be desirable in an SPF 9 product. It is believed that such sunscreen designs are largely implemented for reasons having to do with transparency, as it is believed that consumers will not buy a product which has a chalky appearance, thus reducing the likelihood that consumers will choose to protect themselves against the sun,

In accordance with a preferred embodiment of the invention, chalkiness in the appearance of applied sunscreens is reduced. The same is achieved by using a variety of different materials for the component of a sunscreen formulation which protects against ultraviolet A light. More particularly, in accordance with the invention a blend of white sunscreen material, such as titanium dioxide, is blended with a quantity of colored sunscreen material, such as an iron oxide or iron hydroxide. Because the colored sunscreen material is small enough to be characterized as transparent, it imparts color to the whitish chalky appearance of the other, for example titanium, component.

The result is to reduce the appearance of chalkiness by a tinting effect. At the same time, the amount of, for example, red iron oxide and/or yellow iron oxide, in a formulation is kept at a level which is below that which would provide unacceptable darkening or coloring of the skin. While, the iron oxides do contribute opaque as well as transparent color, the level of the iron oxides is maintained at a low enough value to achieve an acceptable aesthetic appearance.

Generally, it has been discovered that the aesthetic appearance of cosmetic products, such as a sun protecting lotion, sun protecting moisturizer, and sun protecting foundation may be greatly improved while maintaining markedly higher levels of protection to ultraviolet A radiation, by using iron oxides, particularly transparent iron oxides, in the formulation of account of the relatively natural appearance of tinted white sunscreen materials and reflective colored sunscreen materials, provided that the right balance of materials is employed.

The inventive cosmetic products, which may take the form of liquid, crème, stick, compact and other products, are effective products for protecting the skin from sunlight as they contain titanium dioxide and iron oxides of various particle sizes. The inventive products are unlike untinted beach wear sunscreen products, which are expected to be and to varying extents are transparent to visible light in order to avoid undesirable skin whitening. They are also unlike conventional cosmetic products are formulated to provide opacity and cover skin blotches, wrinkles and other imperfections, as well as impart a desirable color and finish to the skin. The inventive products achieve an attractive aesthetic and a large measure of protection by combining what might be regarded as unacceptable amounts of white opacity, colored opacity and colored transparency to provide substantially transparent tinted products having excellent and balanced UV protection.

The cosmetic products contemplated by this invention are color cosmetic products and are typically transparent to modestly opaque products, although they may have lower coverage to make them suitable as make-up primers or cosmetic products for teens. Furthermore, the cosmetic products contemplated by this invention are sunscreen products that provide high amounts of protection against ultraviolet A (UVA) and ultraviolet B (UVB) light.

[TO BE COMPLETED UPON COMPLETION OF CLAIMS]

### DETAILED DESCRIPTION OF THE PREFERRED EMBODIMENTS

An essential component of the cosmetic products contemplated by this invention are that they protect the skin against high amounts of UVA light, which is believed responsible for long-term harm to the skin. The present invention also contemplates the design of cosmetic products with a PA (Protection Against UVA) value of 10.0 or greater measured in-vivo using the Japanese method PPD, (Persistent Pigment Darkening) or an equivalent in-vivo method.

The present invention is also aimed at satisfying the requirements of dermatologists, many of whom are requiring sunscreen products that have high amounts of UVA protection. Currently, the American Academy of Dermatology recommends that a product with an SPF of at least 15 be applied daily. There is presently a strong preference by many dermatologist for sunscreen products with a fixed ratio of UVB to UVA protection (SPF to PA protection). This ratio may be described as the UV balance ratio. The cosmetic products contemplated by this invention allow the achievement of an SPF of at least 30 and a PA of 10 and higher for a UV balance ratio of 3.0 to 1.0 and lower.

Titanium dioxide and iron oxides are available at various particle sizes. During the late 1980's titanium dioxide was typically supplied in three sizes 15 nanometers, 35 nanometers, and pigmentary grades larger than 200 nanometers. Presently, many different particle sizes are available ranging from 10 nm to 300 nm, and even several microns. The titanium dioxide and iron oxide particle sizes contemplated by this invention to attenuate ultraviolet light efficiently and provide UV balance range from about 35 nm or lower to about 60 nm, although other grades may be used outside this range to increase SPF or provide greater opacity.

In accordance with the invention, transparent red iron oxide (Fe₂O₃), transparent yellow iron oxide (Fe₂O(OH)) and transparent black iron oxide (Fe₂O₄) are used in combinations to achieve desired hues and levels of brightness guided by aesthetic considerations moderated by the objective of incorporating a maximum amount of particles with strong UVA attenuation. Particulates are selected keeping in mind UV attenuation. At the same time, the quantities of all particulates are balanced for a pleasant aesthetic effect as noted herein.

An incidental benefit of formulating with iron oxides is UV attenuation, although they are not approved as active sunscreens like titanium dioxide and zinc oxide. Iron oxides provide attenuation against UVA and UVB light, because they scatter and absorb light. Pigmentary iron oxides can contribute more than 0.5 SPF and PA unit per weight percent. The contributions of transparent iron oxides to UV attenuation appear to be greater than for pigmentary grades in cosmetic products. In addition, the aesthetics of the inventive combinations are markedly superior to that achievable with titanium dioxide or zinc oxide based products.

Therefore, the cosmetic products contemplated by this invention seek to provide a PA of at least 10 and a UV balance ratio (UVB:UVA) of 3 to 1 or lower. A likely achievable SPF contemplated by this invention is an SPF of 50 with a PA of 17 and this is believed to be sufficient to protect against sunlight for most commonly encountered circumstances. The cosmetic products contemplated herein attain these high levels of protection using at least 5% of a 35 nm titanium dioxide and roughly about 1.0% or more of a transparent iron oxide, dependent upon the aesthetic result desired. Other particle sizes of titanium dioxides and iron oxides may be added to increase SPF and opacity, similarly, zinc oxide may be added where formulations with less opacity are desired. Larger amounts of UVA attenuating transparent iron oxides will reduce transparency and darken the composition without compromising the acceptability of the aesthetics as appears below.

The following products were formulated using different grades of titanium dioxide to explore the effects of pigment size and pigment content. More particularly, products represent products formulated using dispersions of titanium dioxide containing titanium dioxide particles of different primary particle sizes. A summary of the characteristics of these products is presented below in Table 1. This work is illustrative of potential relationships between titanium dioxide particle size, and the inclusion of iron oxides.

TABLE 1
Exploratory and Inventive Formulations

| | % | Primary Particle | | | Iron | UV | | |
|---|---|---|---|---|---|---|---|---|
| Formula | TiO₂ | Size | SPF | PA | Oxides | Balance | SPF/% TiO₂ | PA/% TiO₂ |
| KLF- | 5.00 | 60 nm | 13.60 | 5.06 | 3.00 | 2.69 | 2.72 | 1.01 |
| 036 | | | | | | | | |
| KLF- | 5.00 | 35 nm | 16.43 | 6.00 | 3.00 | 2.74 | 3.29 | 1.20 |
| 036A | | | | | | | | |
| KSL- | 10.00 | 35 nm | 28.40 | 6.00 | 0.00 | 4.73 | 2.84 | 0.60 |
| 027 | | | | | | | | |
| KSL- | 10.00 | 50 nm | 25.57 | 7.58 | 0.00 | 3.37 | 2.56 | 0.76 |
| 027A | | | | | | | | |
| K2005- | 19.50 | 10 nm, 50 nm, | 32.00 | 15.00 | 3.50 | 2.13 | 1.64 | 0.77 |
| 78 | | 60 nm | | | | | | |

KLF-036 and KLF-036A are aqueous-based products.
KSL-027 and KSL-027A are oil-based products.
In the above table, PA stands for protection against UVA light.
UV balance is the ratio of SPF to PA.
Measurements of PA were taken by Consumer Products Testing Co., an independent laboratory.
Measurements of SPF were also taken by Consumer Products Testing Co.

### Example 1

The ingredients of a first product formulated in accordance with the invention and usable as a moisturizer or sunscreen are shown in Table 2. This product was designated K 2005-76 and was developed with the objective of providing a foundation with high UVA by combining a number of different particle size titanium dioxides and incorporating transparent iron oxides. K 2005-76 was made using a titanium dioxide dispersion sold under catalog number CM3EK25VM by Kobo Products, Inc. This dispersion catalog number CM3EK25VM contains 19.5% active titanium dioxide, by weight that is the weight of the titanium without the weight of surface treatment coatings or the like. K 2005-76 also incorporated a treated titanium dioxide sold under catalog number KQ-MS8 by Kobo Products, Inc. Catalog number KQ-MS8 contains 89.0%, active TiO₂, by weight.

An emollient light ester base, comprising, principally, isononyl isononanoate, silicones, water, waxes and pigments provides moisturization on account of the humectant characteristics of the esters and butylene glycol. A variety of bases to provide moisturization may be contemplated by the present invention. These bases may include any of the materials typically used as bases in prior art cosmetic formulations. The transparent iron oxides noted in Table 2 synergize with the titanium dioxide ingredients to provide an excellent SPF. At the same time, a high PA is also provided in a formulation which has excellent aesthetic appeal. The surface-treated colors, namely the isopropyl titanium triisostearate treated iron oxides in WE55Y, WE70R, and WE70B dispersions deliver smooth application and slip on skin with excellent wear.

Velvesil 125 is used in the inventive formulation to give the product a velvety, cushion-like texture. Naturally, other additives may be used in place of Velvesil 125. The active ingredients of K 2005-76 are the various titanium dioxides which together compromise approximately 19.77% of the final product by weight together with the iron oxides.

TABLE 2
Ingredients of K 2005-76

| SEQ. | | INGREDIENT/ | | SUPPLIER |
|---|---|---|---|---|
| Part | % | TRADE NAME | INCI NAME | NAME |
| 1 | 19.00 | Salacos 99 | isononyl isononanoate | Nisshin Oil M |
| 2 | 2.00 | Lucentite SAN-P | quaternium-18 hectorite | Kobo Products |
| | 1.00 | Ethyl Alcohol SD39C | ethyl alcohol 39C | Warner-Graham |
| | 1.60 | Tarox TRY-100 | yellow iron oxide (20 nm × 100 nm | Kobo Products |
| | | | size) | |
| | 0.70 | Tarox TRR-100 | red iron oxide (20 nm × 100 nm | Kobo Products |
| | | | size) | |
| | 0.05 | Black NF | Iron oxides, 200 nm, cubic shape | Kobo Products |
| | 4.50 | Abil WE09 | polyglyceryl-4 isostearate & cetyl | Goldschmidt |
| | | | PEG/PGG-10/1 dimethicone (and) | |
| | | | hexyl laurate | |
| 3 | 3.00 | Abil WE09 | polyglyceryl-4 isostearate & cetyl | Goldschmidt |
| | | | PEG | |
| | | | PGG-10/1 dimethicone (and) | Goldschmidt |
| | | | hexyl laurate | |
| | 0.50 | Abil Wax 9801 | cetyl dimethicone | Goldschmidt |
| | 18.90 | TiO₂ KQ-MS8 | Titanium dioxide (and) aluminum | Kobo Products |
| | | | dydroxide (and) methicone; size is | |
| | | | 60 nm alumina coated. | |
| | 15.12 | CM3EK25VM | cyclopentasiloxane (and) ethyl | Kobo Products |
| | | | trisiloxane (and) titanium dioxide | |
| | | | (and) methicone (and) PEG-10 | |
| | | | dimethicone; size is 10 nm | |
| | | | acicular | |
| | 2.00 | WE55Y | yellow iron oxide (C.I. 77491) | Kobo Products |
| | | | (and_) polyglyceryl-4 isostearate | |
| | | | (and) cetyl PEG/PGG-10/1 | |
| | | | dimethicone (and) hexyl laurate | |
| | | | (and) isopropyl titanium | |
| | | | triisostearate; size is 370-400 nm | |
| | 0.40 | WE70R | red iron oxide (C.I. 77492) (and) | Kobo Products |
| | | | polyglyceryl-4 isostearate (and) | |
| | | | cetyl PEG/PGG-10/1 dimethicone | |
| | | | (and) hexyl laurate (and) isopropyl | |
| | | | titanium triisostearate; particle size | |
| | | | is 70-100 nm | |
| 4 | 0.75 | Crill 6 | sorbitan isosterate | Croda |
| 5 | 12.50 | Water | water | Symrise |
| | 1.00 | Symdiol 68 | 1,2-hexandiol (and) 1,2-octanediol | Axo Chemical |
| | 2.50 | Butylene Glycol | butylenes glycol | Morton Salt |
| 6 | 1.00 | Sodium Chloride | sodium chloride | Strahl & Pitsch |
| | 1.65 | Syncrowax HGL-C | C18-36 griglycerides | Croda |
| 7 | 9.98 | Velvesil 125 | Cyclopentasiloxane (and) C30-45 | GES/Kobo |
| | | | alkyl cetearyl dimethicone | |
| | | | crosspolymer | |
| 8 | 0.75 | Phenonip XB | Phenoxyethanol & methylparaben | Nipa Products |
| | | | propylparaben & ethylparaben | |

### Procedure For Manufacture of K 2005-76

A first phase is formulated by slowly adding quaternium-18 hectorite to isononyl isononanoate while stirring with a Cowles brand dissolver in a stainless steel beaker at high speed for 20 minutes. The ethyl alcohol 39C is added to the first phase and stirred for an additional 20 minutes. Ethyl alcohol 39C is USP grade of ethyl alcohol. The mixture is then set aside. In time, the same develops into a lucentite gel.

In a separate stainless steel beaker, the 4.50% quantity of Polyglyceryl-4 isostearate and cetyl PEG/PGG-10/1 dimethicone (and) hexyl laurate (Abil WE09) are combined with the transparent iron oxides (product names Tarox TRY-100, Tarox TRR-100, Black NF) to produce phase 2. The mixture is dispersed for 90 minutes using a dispersator, such as a dispersator manufactured by Morehouse, Cowles or Premier Mill Co.

The color particles, namely the transparent iron oxides, are then checked under a microscope. The mixture is looked at to determine if the pigments are fully dispersed. A satisfactory result is indicated by a uniform small appearance of the pigments and the absence of large agglomerates. This indicates that the iron oxide slurry is well dispersed.

When a satisfactory iron oxide slurry has been achieved, the lucentite gel (or, alternatively, any organophilic gel such as versagel, bentones, lucentite or any suitable smectite clay or polymer gel) of phase 1 is combined with the transparent iron oxide slurry of phase 2. The same is stirred at high speed for 30 minutes with a dispersator (such as the Cowles dissolver) until the mixture is homogenous to form a pre-formed color gel phase.

Next, the Polyglyceryl-4 isostearate and cetyl PEG/PGG-10/1 dimethicone and hexyl laurate cetyl dimethicone dispersion; the titanium dioxide, aluminum hydroxide and methicone dispersion; the dispersion of cyclopentasiloxane, ethyl trisiloxane, titanium dioxide, methicone and PEG-10 dimethicone; the dispersion of iron oxide (C.I. 77491) in polyglyceryl-4 isostearate, cetyl PEG/PGG-10/1 dimethicone, hexyl laurate and isopropyl titanium triisostearate; iron oxide (C.I. 77492), polyglyceryl-4 isostearate, cetyl PEG/PGG-10/1 dimethicone, hexyl laurate and isopropyl titanium triisostearate; and Iron Oxide (C.I. 77499) dispersed in polyglyceryl-4 isostearate (and) cetyl PEG/PGG-10/1 dimethicone, hexyl laurate and isopropyl titanium triisostearate of Part 3 are combined in a stainless steel beaker. In the above, C.I. refers to Color Index Number.

Phase 3 is then stirred with a Cowles brand dissolver for 5 minutes. One then slowly adds the pre-formed color gel phase, made by combining phase 1 and phase 2 to phase 3 to form the base. The base is then stirred with a Cowles brand dissolver for 5 minutes.

The sorbitan isosterate of Part 4 is added to the base and mixes for an additional 10 minutes at a high speed with a Cowles brand dissolver. Mixing is then continued for an additional 60 minutes while heating to a temperature in the range of about 60-65 degrees Celsius.

One then adds the waxes, namely the microcrystalline wax and C18-36 triglycerides of Part 6 to the mixture of Part 4, which is maintained at about 65 degrees Celsius. This is stirred for five minutes. The aqueous ingredients of Part 5 are combined and stirred until clear. The mixture is then emulsified with the dispersator while heating to 82 degrees Celsius. During this part of the method, the beaker is kept covered.

After reaching 82 degrees Celsius, the mixture is mixed for an additional five minutes. Such mixing may be done with the Cowles dispersator. The material is removed from the Cowles dissolver and then mixed with a Silverson at 8000 rpm using the largest screen supplied in a steam bath.

Air cooling while mixing is continued for the purpose of homogenizing to form a uniform emulsion. When the mixture reaches 70 degrees Celsius, the mixture of cyclopentasiloxane and C30-45 alkyl cetearyl dimethicone crosspolymer (Velvesil) of Part 7 is added. When the mixture reaches 65 degrees Celsius, the mixture of Part 8, henoxyethanol and methylparaben, and of propylparaben and ethylparaben is added. One then continues homogenizing in the Silverson until the mixture cools down to 25-30 degrees Celsius. It is then introduced into appropriate containers. In vivo tests conducted for SPF using the FDA Monograph, Static Efficacy and for PFA using the JCIA Persistent Pigment Darkening (PPD) yielded the results shown in Tables 3 and 4.

TABLE 3
Results for K 2005-76
Individual SPF Values

| | | | | 8% | |
|---|---|---|---|---|---|
| | | | | homosalate | |
| | | | | (HMS) | K2005-76 UV |
| Subject | Skin | | | Standard | Balance Foundation |
| Number | Type | Age | Sex | SPF | LMU |
| 1 | II | 60 | F | 4.4 | 32.1 |
| 2 | III | 48 | F | 6.6 | 30.0 |
| 3 | III | 55 | M | 5.0 | 32.1 |

| | | |
|---|---|---|
| Average SPF | 5.33 | 31.40 |

I - very fair Caucasian
II - fair Caucasian
III - normal Caucasian
IV - Hispanic and Asian skin tone

TABLE 4
Results for K 2005-76
Individual PFA Values

| | | | | Test duration: 180 Minutes | |
|---|---|---|---|---|---|
| | | | | | K2005-76 UV |
| Subject | Skin | | | | Balance Foundation |
| Number | Type | Age | Sex | Standard | Liquid Makeup (LMU) |
| 1 | III | 43 | F | 3.75 | 12.51 |
| 2 | IV | 47 | M | 4.69 | 15.61 |
| 3 | III | 47 | M | 3.75 | 12.49 |

| | | |
|---|---|---|
| Average PFA (n = 3) | 4.06 | 13.54 |

### Example 2

The ingredients of a second product, K 2005-78, are shown in Table 5. This product was designated K 2005-78 and was developed with the objective of providing a foundation with high UVA by combining a number of different particle size titanium dioxides and incorporating transparent iron oxides. K 2005-78 was made using a titanium dioxide dispersion sold under catalog number CM3EK25VM by Kobo Products, Inc. This dispersion, catalog number CM3EK25VM, contains 19.5% active titanium dioxide, by weight that is the weight of the titanium without the weight of surface treatment coatings or the like. K 2005-78 also incorporated a treated titanium dioxide sold under catalog number KQ-MS8 by Kobo Products, Inc. Catalog number KQ-MS8 contains 89.0%, active titanium dioxide, by weight. K 2005-78 also incorporated a treated titanium dioxide sold under catalog number MT-600B-MS7 by Kobo Products, Inc. Catalog number MT-600B-MS7 contains 93.0%, active titanium dioxide, by weight.

Using the above method steps described with respect to Example 1, the ingredients shown in Table 5 were combined to make a cosmetic foundation.

TABLE 5
Ingredients of K2005-78

| SEQ. | | INGREDIENT/ | | SUPPLIER |
|---|---|---|---|---|
| Part | % | TRADE NAME | INCI NAME | NAME |
| 1 | 19.00 | Salacos 99 | isononyl isononanoate | Nisshin Oil |
| | | | | Mills |
| 2 | 2.00 | Lucentite SAN-P | quaternium-18 hectorite | Kobo Products |
| | 1.00 | Ethyl Alcohol | ethyl alcohol 39C | Warner- |
| | | SD39C | | Graham |
| | 1.60 | Tarox TRY-100 | yellow iron oxide, 20 nm × 100 nm | Kobo Products |
| | | | size | |
| | 0.70 | Tarox TRR-100 | red iron oxide, 20 nm × 100 nm size | Kobo Products |
| | 0.05 | Black NF | black iron oxides, 200 nm, cubic | Kobo Products |
| | | | shape | |
| | 4.50 | Abil WE09 | polyglyceryl-4 isostearate & cetyl | Goldschmidt |
| | | | PEG/ | |
| | | | PGG-10/1 dimethicone (and) hexyl | |
| | | | laurate | |
| 3 | 3.00 | Abil WE09 | polyglyceryl-4 isostearate & cetyl | Goldschmidt |
| | | | PEG/ | |
| | | | PGG-10/1 dimethicone (and) hexyl | |
| | | | laurate | |
| | 0.50 | Abil Wax 9801 | cetyl dimethicone | Goldschmidt |
| | 12.45 | TiO₂ KQ-MS8 | titanium dioxide (and) aluminum | Kobo Products |
| | | | hydroxide (and) methicone | |
| | 6.23 | MT-600B-MS7 | titanium dioxide (and) methicone, 50 nm | Kobo Products |
| | | | TiO₂; coated with methicone by | |
| | | | Kobo | |
| | 14.94 | CM3EK25VM | cyclopentasiloxane (and) ethyl | Kobo Products |
| | | | trisiloxane | |
| | | | (and) titanium dioxide (and) | |
| | | | methicone | |
| | | | (and) PEG-10 dimethicone | |
| | 2.00 | WE55Y | iron oxide (C.I. 77491) (and) | Kobo Products |
| | | | polyglyceryl-4 | |
| | | | isostearate (and) cetyl PEG/PGG- | |
| | | | 10/1 | |
| | | | dimethicone (and) hexyl laurate (and) | |
| | | | isopropyl titanium triisostearate | |
| | 0.40 | WE70R | iron oxide (C.I. 77492) (and) | Kobo Products |
| | | | polyglyceryl-4 | |
| | | | isostearate (and) cetyl PEG/PGG- | |
| | | | 10/1 | |
| | | | dimethicone (and) hexyl laurate (and) | |
| | | | isopropyl titanium triisostearate | |
| | 0.10 | WE70B | Black iron oxide (C.I. 77499) (and) | Kobo Products |
| | | | polyglyceryl-4 | |
| | | | isostearate (and) cetyl PEG/PGG- | |
| | | | 10/1 | |
| | | | dimethicone (and) hexyl laurate (and) | |
| | | | isopropyl titanium triisostearate; | |
| | | | particle size 160-280 nm | |
| 4 | 0.75 | Crill 6 | sorbitan isostearate | Croda |
| 5 | 12.50 | Water | Water | |
| | 1.00 | Symdiol 68 | 1,2-hexandiol (and) 1,2-octanediol | Symrise |
| | 2.50 | Butylene Glycol | butylene glycol | Axo Chemical |
| | 1.00 | Sodium Chloride | sodium chloride | Morton Salt |
| 6 | 1.00 | Microcrystalline | microcrystalline wax | Strahl & Pitsch |
| | | SP89 | | |
| | 1.65 | Syncrowax HGL-C | Triglycerides | Croda |
| | | C18-36 | | |
| 7 | 10.38 | Velvesil 125 | cyclopentasiloxane (and) C30-45 | GES/Kobo |
| | | | alkylCetearyl dimethicone | |
| | | | crosspolymer | |
| 8 | 0.75 | Phenonip XB | phenoxyethanol and methylparaben | Nipa Products |
| | | | propylparaben and ethylparaben | |
| | 100.00 | | | |

The mixing method of Example 1 for the various parts was employed to make this product, as well as the other products noted below. SPF and protection from the daylight was measured with the results shown in Tables 6 and 7.

TABLE 6
Results for K 2005-78
Individual SPF Values

| | | | | | K2005-78 UV |
|---|---|---|---|---|---|
| Subject | Skin | | | 8% HMS | Balance Foundation |
| Number | Type | Age | Sex | Standard | LMU |
| I | II | 60 | M | 5.0 | 30.0 |
| 2 | III | 45 | M | 4.4 | 34.5 |
| 3 | II | 51 | F | 5.0 | 30.0 |

| | | |
|---|---|---|
| Average SPF | 4.80 | 31.50 |

TABLE 7
Individual PFA Values

| | | | | 180 Minutes | |
|---|---|---|---|---|---|
| | | | | | K2005-78 UV |
| Subject | Skin | | | | Balance Foundation |
| Number | Type | Age | Sex | Standard | LMU |
| 4 | III | 48 | F | 4.69 | 15.62 |
| 5 | IV | 48 | M | 3.75 | 15.63 |
| 6 | IV | 36 | M | 4.68 | 15.62 |

| | | |
|---|---|---|
| Average PFA (n = 3) | 4.37 | 15.62 |

### Example 3

The ingredients of a third product are shown in Table 8. This product was designated K 2005-80 and was developed with the objective of providing a foundation with high UVA by combining a number of different particle size titanium dioxides and incorporating transparent iron oxides. K 2005-80 was made using a titanium dioxide dispersion sold under catalog number CM3EK25VM by Kobo Products, Inc. This dispersion catalog number CM3EK25VM contains 19.5% active titanium dioxide, by weight that is the weight of the titanium without the weight of surface treatment coatings or the like. K 2005-80 also incorporated a treated titanium dioxide sold under catalog number KQ-MS8 by Kobo Products, Inc. Catalog number KQ-MS8 contains 89.0%, active titanium dioxide, by weight. K 2005-80 also incorporated a treated titanium dioxide sold under catalog number MT-600B-MS7 by Kobo Products, Inc. Catalog number MT-600B-MS7 contains 93.0%, active titanium dioxide, by weight. K 2005-80 also incorporated a treated titanium dioxide sold under catalog number MT-500H-11S5 by Kobo Products, Inc. Catalog number MT-500H-11S5 contains 90.0%, active titanium, by weight.

Using the above method steps described with respect to Example 1, the ingredients shown in Table 8 were combined to make a cosmetic foundation.

TABLE 8
Ingredients of K2005-80

| SEQ. | | | | |
|---|---|---|---|---|
| NAME - | | INGREDIENT/ | | |
| Part | % | TRADE NAME | INCI NAME | SUPPLIER |
| 1 | 19.00 | Salacos 99 | isononyl isononanoate | Nisshin Oil |
| | | | | Mills |
| 2 | 2.00 | Lucentite SAN-P | quaternium-18 hectorite | Kobo Products |
| | 1.00 | Ethyl Alcohol SD39C | ethyl alcohol 39C | Warner- |
| | | | | Graham |
| | 1.60 | Tarox Try-100 | yellow iron oxide, 20 nm × 100 nm | Kobo Products |
| | | | size | |
| | 0.70 | Tarox TRR-100 | red iron oxide, 20 nm × 100 nm size | Kobo Products |
| | 0.05 | Black NF | black iron oxides, 200 nm, cubic | Kobo Products |
| | | | shape | |
| | 4.50 | Abil WE09 | polyglyceryl-4 isostearate & cetyl | Goldschmidt |
| | | | PEG/PGG-10/1 dimethicone (and) | |
| | | | hexyl laurate | |
| 3 | 3.00 | Abil WE09 | polyglyceryl-4 isostearate & cetyl | Goldschmidt |
| | | | PEG/PGG-10/1 dimethicone (and) | |
| | | | hexyl laurate | |
| | 0.50 | Abil Wax 9801 | cetyl dimethicone | Goldschmidt |
| | 6.20 | TiO₂ KQ-MS8 | titanium dioxide (and) aluminum | Kobo Products |
| | | | hydroxide (and) methicone | |
| | 6.20 | MT-600B-MS7 | titanium dioxide (and) methicone, | Kobo Products |
| | | | 50 nm TiO₂, coated with methicone | |
| | | | by Kobo | |
| | 6.20 | MT-500H-11S5 | titanium dioxide (and) alumina (and) | Kobo Products |
| | | | triethoxycaprylylsilane; 35 nm | |
| | | | TiO₂, coated with alumina by | |
| | | | supplier and coated with methicone | |
| | | | by Kobo | |
| | 14.88 | CM3EK25VM | cyclopentasiloxane (and) ethyl | Kobo Products |
| | | | trisiloxane (and) titanium dioxide | |
| | | | (and) methicone (and) PEG-10 | |
| | | | dimethicone | |
| | 2.00 | WE55Y | iron oxide (C.I. 77491) (and) | Kobo Products |
| | | | polyglyceryl-4 isostearate (and) | |
| | | | cetyl PEG/PGG-10/1 dimethicone | |
| | | | (and) hexyl laurate (and) isopropyl | |
| | | | titanium triisostearate | |
| | 0.40 | WE70R | iron oxide (C.I. 77492) (and) | Kobo Products |
| | | | polyglyceryl-4 isostearate (and) | |
| | | | cetyl PEG/PGG-10/1 dimethicone | |
| | | | (and) hexyl laurate (and) isopropyl | |
| | | | titanium triisostearate | |
| | 0.10 | WE70B | iron oxide (C.I. 77499) (and) | Kobo Products |
| | | | polyglyceryl-4 isostearate (and) | |
| | | | cetyl PEG/PGG-10/1 dimethicone | |
| | | | (and) hexyl laurate (and) isopropyl | |
| | | | titanium triisostearate | |
| 4 | 0.75 | Crill 6 | sorbitan isostearate | Croda |
| 5 | 12.50 | Water | Water | |
| | 1.00 | Symdiol 68 | 1,2-hexandiol (and) 1,2-octanediol | Symrise |
| | 2.50 | Butylene Glycol | butylene glycol | Axo Chemical |
| | 1.00 | Sodium Chloride | sodium chloride | Morton Salt |
| 6 | 1.00 | Microcrystalline | microcrystalline wax | Strahl & Pitsch |
| | | SP89 | | |
| | 1.65 | Syncrowax HGL-C | C18-36 triglycerides | Croda |
| 7 | 10.52 | Velvesil 125 | cyclopentasiloxane (and) C30-45 | GES/Kobo |
| | | | alkyl cetearyl dimethicone | |
| | | | crosspolymer | |
| 8 | 0.75 | Phenonip XB | phenoxyethanol & methylparaben | Nipa Products |
| | | | propylparaben & pthylparaben | |
| | 100.00 | | | |

SPF and protection from the daylight was measured with the results shown in Tables 9 and 10.

TABLE 9
Results for K 2005-80
Individual SPF Values

| | | | | | K2005-80 |
|---|---|---|---|---|---|
| Subject | Skin | | | 8% HMS | Balance Foundation |
| Number | Type | Age | Sex | Standard | LMU |
| 1 | II | 44 | M | 5.0 | 34.5 |
| 2 | III | 43 | M | 5.0 | 32.1 |
| 3 | II | 43 | F | 4.4 | 34.5 |

| | | |
|---|---|---|
| Average SPF | 4.80 | 33.71 |

TABLE 10
Individual PFA Values

| | | | | 180 Minutes | |
|---|---|---|---|---|---|
| | | | | | K2005-80 UV |
| Subject | Skin | | | | Balance Foundation |
| Number | Type | Age | Sex | Standard | LMU |
| 1 | III | 21 | M | 3.00 | 12.50 |
| 2 | IV | 30 | F | 4.69 | 15.62 |
| 3 | III | 36 | F | 3.75 | 12.49 |

| | | |
|---|---|---|
| Average PFA | 3.81 | 13.54 |

### Example 4

The ingredients of a forth product are shown in Table 11. This product was designated K 2005-82 and was developed with the objective of providing a foundation with high UVA protection by combining a number of different particle size titanium dioxides and incorporating transparent iron oxides. K 2005-82 was made using a titanium dioxide dispersion sold under catalog number CM3EK25VM by Kobo Products, Inc. This dispersion catalog number CM3EK25VM contains 19.5% active titanium dioxide, by weight that is the weight of the titanium without the weight of surface treatment coatings or the like. K 2005-82 also incorporated a treated titanium dioxide sold under catalog number KQ-MS8 by Kobo Products, Inc. Catalog number KQ-MS8 contains 89.0%, active TiO₂, by weight. K 2005-82 also incorporated a treated titanium dioxide sold under catalog number MT-600B-MS7 by Kobo Products, Inc. Catalog number MT-600B-MS7 contains 93.0%, active TiO₂, by weight. K 2005-82 also incorporated a treated titanium dioxide sold under catalog number MT-500B-11S5 by Kobo Products, Inc. Catalog number MT-500B-11S5 contains 95.0%, active TiO₂, by weight.

Using the above method steps described with respect to Example 1, the ingredients shown in Table 11 were combined to make a cosmetic foundation.

TABLE 11
Ingredients of K2005-82

| SEQ. | | | | |
|---|---|---|---|---|
| NAME | | INGREDIENT/ | | |
| Part | % | TRADE NAME | INCI NAME | SUPPLIER |
| 1 | 19.00 | Salacos 99 | isononyl isononanoate | Nisshin Oil |
| | | | | Mills |
| 2 | 2.00 | Lucentite SAN-P | Quaternium-18 hectorite | Kobo Products |
| | 1.00 | Ethyl Alcohol SD39C | Ethyl alcohol 39C | Warner- |
| | | | | Graham |
| | 1.60 | Tarox TRY-100 | yellow iron oxide, 20 nm × 100 nm | Kobo Products |
| | | | size | |
| | 0.70 | Tarox TRR-100 | red iron oxide, 20 nm × 100 nm size | Kobo Products |
| | 0.05 | Black NF | black iron oxide, 200 nm, cubic | Kobo Products |
| | | | shape | |
| | 4.50 | Abil WE09 | polyglyceryl-4 isostearate & cetyl | Goldschmidt |
| | | | PEG/PGG-10/1 dimethicone (and) | |
| | | | hexyl laurate | |
| 3 | 3.00 | Abil WE09 | polyglyceryl-4 isostearate & cetyl | Goldschmidt |
| | | | PEG/PGG-10/1 dimethicone (and) | |
| | | | hexyl laurate | |
| | 0.50 | Abil Wax 9801 | cetyl dimethicone | Goldschmidt |
| | 6.26 | TiO₂ KQ-MS8 | titanium dioxide (and) aluminum | Kobo Products |
| | | | hydroxide (and) methicone | |
| | 6.26 | MT-600B-MS7 | titanium dioxide (and) methicone; | Kobo Products |
| | | | 50 nm TiO₂, coated with methicone | |
| | | | by Kobo | |
| | 6.26 | MT-500B-11S5 | titanium dioxide (and) | Kobo Products |
| | | | triethoxycaprylylsilane; 35 nm | |
| | | | TiO₂; coated with alumina by | |
| | | | supplier and coated with methicone | |
| | | | by Kobo; and 35 nm TiO₂ coated | |
| | | | with silane by Kobo | |
| | 12.53 | CM3EK25VM | cyclopentasiloxane (and) ethyl | Kobo Products |
| | | | trisiloxane (and) titanium dioxide | |
| | | | (and) methicone (and) PEG-10 | |
| | | | dimethicone | |
| | 2.00 | WE55Y | iron oxide (C.I. 77491) (and) | Kobo Products |
| | | | polyglyceryl-4 isostearate (and) | |
| | | | cetyl PEG/PGG-10/1 dimethicone | |
| | | | (and) hexyl laurate (and) isopropyl | |
| | | | titanium triisostearate | |
| | 0.40 | WE70R | iron oxide (C.I. 77492) (and) | Kobo Products |
| | | | polyglyceryl-4 isostearate (and) | |
| | | | cetyl PEG/PGG-10/1 dimethicone | |
| | | | (and) hexyl laurate (and) isopropyl | |
| | | | titanium triisostearate | |
| | 0.10 | WE70B | iron oxide (C.I. 77499) (and) | Kobo Products |
| | | | polyglyceryl-4 isostearate (and) | |
| | | | cetyl PEG/PGG-10/1 dimethicone | |
| | | | (and) hexyl laurate (and) Isopropyl | |
| | | | titanium triisostearate | |
| 4 | 0.75 | Crill 6 | sorbitan isostearate | Croda |
| 5 | 12.50 | Water | Water | |
| | 1.00 | Symdiol 68 | 1,2-hexandiol (and) 1,2-octanediol | Symrise |
| | 2.50 | Butylene Glycol | butylene glycol | Axo Chemical |
| | 1.00 | Sodium Chloride | sodium chloride | Morton Salt |
| 6 | 1.00 | Microcrystalline | microcrystalline wax | Strahl & Pitsch |
| | | SP89 | | |
| | 1.65 | Syncrowax HGL-C | C18-36 triglycerides | Croda |
| 7 | 12.69 | Velvesil 125 | cyclopentasiloxane (and) C30-45 | GES/Kobo |
| | | | alkyl cetearyl dimethicone | |
| | | | crosspolymer | |
| 8 | 0.75 | Phenonip XB | phenoxyethanol and methylparaben | Nipa Products |
| | | | propylparaben and ethylparaben | |
| | 100.00 | | | |

SPF and protection from the day light was measured with the results shown in Tables 12 and 13.

TABLE 12
Results for K 2005-82
Individual SPF Values

| | | | | | K2005-82 UV |
|---|---|---|---|---|---|
| Subject | Skin | | | 8% HMS | Balance Foundation |
| Number | Type | Age | Sex | Standard | LMU |
| 1 | II | 43 | M | 4.4 | 34.5 |
| 2 | II | 61 | M | 4.4 | 30.0 |
| 3 | III | 57 | F | 4.4 | 32.1 |

| | | |
|---|---|---|
| Average SPF | 4.40 | 32.20 |

TABLE 13
Individual PFA Values

| | | | | 180 Minutes | |
|---|---|---|---|---|---|
| | | | | | K2005-82 UV |
| Subject | Skin | | | | Balance Foundation |
| Number | Type | Age | Sex | Standard | LMU |
| 1 | * | * | * | * | * |
| 2 | III | 27 | F | 3.75 | 12.50 |
| 3 | III | 41 | F | 3.75 | 15.63 |
| 4 | III | 31 | F | 3.75 | 12.51 |

| | | |
|---|---|---|
| Average PFA (n = 3) | 3.75 | 13.55 |

* Data not included in calculations - Panelist noncompliant - Data rejected.

The constituents of the ingredients in the examples are detailed below in Table 14.

TABLE 14
Compositional Breakdown for
Inventive Composition (Weight Percent)

| | INGREDIENT | | | | | | | | |
|---|---|---|---|---|---|---|---|---|---|
| | | | MT- | MT- | MT- | TiO2 | | | |
| | CM3EK- | BLACK | 600B- | 500H- | 500B- | KQ- | | | |
| | 25VM | NF | MS7 | 11S5 | 11S5 | MS8 | WE55Y | WE70R | WE70B |
| Constituents of | | — | — | — | — | — | — | — | — |
| Ingredients | | | | | | | | | |
| Cyclopenta- | 35-39 | — | — | — | — | — | — | — | — |
| siloxane | | | | | | | | | |
| Ethyl Trisiloxane | 24-28 | — | — | — | — | — | — | — | — |
| Titanium | 17-22 | — | 91-95 | 88.4-92 | 94-96 | 85-90 | — | — | — |
| Dioxide | | | | | | | | | |
| PEG-10 | 10-14 | — | — | — | — | — | — | — | — |
| Dimethicone | | | | | | | | | |
| Alumina | 2-5 | — | — | 3-6.6 | — | — | — | — | — |
| Methicone | 0.5-2.5 | — | 6-8 | — | — | 6-9 | — | — | — |
| Iron Oxides | — | — | — | — | — | — | 52-54.5 | 67-69 | — |
| (C.I. 77491) | | | | | | | | | |
| Iron Oxides | — | — | — | — | — | — | — | — | 67-69 |
| (C.I. 77492) | | | | | | | | | |
| Iron Oxides | — | 95-100 | — | — | — | — | — | — | — |
| (C.I. 77499) | | | | | | | | | |
| Triethoxycaprylylsilane | — | — | — | 4-6 | 4-6 | — | — | — | — |
| Aluminum | — | — | — | — | — | 1-4 | — | — | — |
| Hydroxide | | | | | | | | | |
| Hexyl | — | — | — | — | — | — | 13-17 | 8-12 | 8-12 |
| Laurate | | | | | | | | | |
| Cetyl | — | — | — | — | — | — | 13-17 | 8-12 | 8-12 |
| PEG/PPG-10/1 | | | | | | | | | |
| Dimethicone | | | | | | | | | |
| Polyglyceryl-4 | — | — | — | — | — | — | 13-17 | 8-12 | 8-12 |
| Isostearate | | | | | | | | | |
| Isopropyl | — | — | — | — | — | — | .90-1.3 | 1.2-1.6 | 1.2-1.6 |
| Titanium | | | | | | | | | |
| Triisostearate | | | | | | | | | |

In accordance with the present invention, a dispersion for achieving cosmetic products of the type of the present invention may also be provided. Such dispersion may be used in place of conventional dispersions in otherwise conventional product recipes. It is contemplated in accordance with the invention that conventional dispersion manufacturing techniques may be used to incorporate particulates into a dispersion. A typical formulation for such a dispersion is given in Table 15. The particulates used may be hydrophobic or hydrophilic and may be incorporated in water or oil vehicles as is known in the prior art for such materials.

For example, such a formulation may be achieved by putting solvents (in an amount of approximately equal in weight to the weight of the particulates) such as cyclopentasiloxane and ethyl trisoloxane in a mixing tank. Next, a surfactant is dissolved in the solvents. Lastly, the particulates as detailed in Table 15 are headed. The particulates are then mixed with the surfactant and solvent at approximately 500 rpm with the disburse or blade. The mixture is then transferred to a mixing tank and subsequently milled using a bead mill to the desired particle size.

If desired, a dispersant such as polyhydroxystearic acid may be used without the need for using hydrophobized particulates, despite the use of a nonaqueous vehicle. Alternatively, hydrophobized particulates may be used in an oil, or other nonaqueous vehicle. The incorporation of the particulates may also be made into an aqueous base, or a silicone based carrier vehicle.

TABLE 15
Particulate Ingredients for Dispersion

| | INGREDIENT/ | | SUPPLIER |
|---|---|---|---|
| Parts | TRADE NAME | INCI NAME | NAME |
| 16 | Tarox TRY-100 | yellow iron oxide, 20 nm × | Kobo |
| | | 100 nm size | Products |
| 7 | Tarox TRR-100 | red iron oxide, 20 nm × 100 nm | Kobo |
| | | size | Products |
| .5 | Black NF | black iron oxides, 200 nm, cubic | Kobo |
| | | shape | Products |
| 110 | | 60 nm titanium dioxide | |
| 55 | | 50 nm titanium dioxide | |
| 30 | | 10 nm titanium | |
| 10 | | 370-400 nm yelow iron oxide | |
| | | (C.I. 77491) | |
| 2.8 | | 70-100 iron oxide (C.I. 77492) | |
| .7 | | black iron oxide (C.I. 77499); | |
| | | particle size 160-280 nm | |

1-39. (canceled)

40. A cosmetic composition comprising a transparent substantially colorless pigment, said substantially colorless pigment comprising a first group of particles sufficiently small to be substantially transparent to visible light and substantially opaque to ultraviolet B light, said substantially colorless pigment further comprising a second group of particles sufficiently small to be minimally reflective to visible light and substantially opaque to ultraviolet A light whereby said second group of particles results in minimal visible reflection by the cosmetic composition; and a substantially transparent coloring pigment, said coloring pigment substantially transmitting visible light and reflecting a substantial portion of incident ultraviolet A light to impart color to tint visible light reflected by the cosmetic composition when said cosmetic composition is disposed over the skin.

41. A cosmetic composition as in claim 1, further comprising a carrier vehicle.

42. A cosmetic composition as in claim 2, wherein said cosmetic composition has a protection against UV factor (PA) which is one third the sun protection factor (SPF) of said cosmetic composition or greater, and an SPF greater than 20.

43. A cosmetic composition as in claim 2, wherein said substantially colorless pigment is selected from the group consisting of titanium dioxide and zinc oxide; and said coloring pigment is selected from the group consisting of red iron oxide, black iron oxide and yellow iron oxide.

44. A cosmetic composition as in claim 4, wherein said first group of particles comprises titanium dioxide having a mean particle size ranging between 5 nm and 20 nm, and said second group of particles comprises titanium dioxide having a mean particle size ranging between 40 nm and 65 nm and said second group of particles comprises a first subgroup of particles having a mean particle size ranging between 45 nm and 55 nm, and a second subgroup of particles having a mean particle size ranging between 55 nm and 65 nm.

45. A cosmetic composition as in claim 5, wherein said cosmetic composition has a protection against UV factor (PA) which is one third the sun protection factor (SPF) of said cosmetic composition or greater, and an SPF greater than 20.

46. A cosmetic composition as in claim 5, wherein said iron oxides comprise iron oxides which range in size between 10 nm and 30 nm.

47. A cosmetic composition as in claim 7, wherein said iron oxides further comprise additional iron oxides which range in size between 50 nm and 120 nm.

48. A cosmetic composition as in claim 5, or insert first group of particles comprises between 12% and 20% of said titanium dioxide wherein first subgroup of particles comprises between 20% and 35% of said titanium dioxide, and said second subgroup of particles comprises between 40% and 70% of said titanium dioxide.

49. A cosmetic composition as in claim 2, wherein said first group of particles comprises between 12% and 20% of said colorless pigment and wherein said second group of particles comprises a first subgroup of particles comprising between 20% and 35% of said colorless pigment, and a second subgroup of particles comprising between 40% to 70% of said colorless pigment and wherein said first group of particles has a mean particle size ranging between 7 nm and 13 nm, said first subgroup of particles has a mean particle size ranging between 47 nm and 53 nm, and said second subgroup of particles has a mean particle size ranging between 58 nm and 62 nm.

50. A cosmetic composition as in claim 2, wherein said substantially colorless pigment comprises between 3 and 7 percent by weight of the composition and said transparent coloring pigment comprises between 0.5 and 1.5% of the composition by weight.

51. A cosmetic product as in claim 1, wherein said cosmetic product as claimed in claim 1 comprises about 10% or more of a titanium dioxide pigment with a primary particle size of about 35 nm to 60 nm.

52. A method using the material of claim 1, to make a liquid foundation.

53. A cosmetic product as in claim 2, wherein the carrier holds the other pigments in an emulsion.

54. A dispersion comprising a transparent substantially colorless pigment, said substantially colorless pigment comprising a first group of particles sufficiently small to be substantially transparent to visible light and substantially opaque ultraviolet B light, said substantially colorless pigment further comprising a second group of particles sufficiently small to be minimally reflective to visible light and substantially opaque to ultraviolet A light whereby said second group of particles results in minimal visible reflection by the cosmetic composition; a substantially transparent coloring pigment, said coloring pigment substantially transmitting visible light and reflecting a substantial portion of incident ultraviolet A light to impart color to tint visible light reflected by the cosmetic composition when said cosmetic composition is in use; and a carrier vehicle.

55. A cosmetic product containing titanium dioxide and iron oxides with a PA of about 10 or greater and an SPF equal to or three times greater than the PA result up to SPF 50.
